(19) United States
(12) Patent Application Publication
(10) Pub. No.: US 2006/0009463 A1
(43) Pub. Date: Jan. 12, 2006

(54) N-(3-rifamycinyl)-carbamates, method of preparing them and their use for treating and preventing tuberculosis

(76) Inventors: Dimcho Ivanov Dimov, Razgrad (BG); Nely Tzoneva Angelova, Razgrad (BG); Asenov Grozdanov, Razgrad (BG); Pavel Nenov Penev, Razgrad (BG); Svetlana Tzvetkova Haladgova, Razgrad (BG); Desislava Todorova Mincheva, Razgrad (BG); Stevka Tzvetkova Paraskevova, Razgrad (BG); Sashko Ivanov Dekin, Razgrad (BG); Iskra Todorova Atanasova, Razgrad (BG); Gerrit Schuurmann, Threna (DE); Ralph Kuhne, Halle (DE); Peter William Andrew, Leicester (GB); Jamilia Shafi, Leicester (GB); Jason Anthony Sharpe, Oldsawley (GB)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/510,577

(22) PCT Filed: Apr. 10, 2003
(86) PCT No.: PCT/EP03/03751

§ 371 Date: Jul. 15, 2005

(30) Foreign Application Priority Data
Apr. 10, 2002 (DE) 102 16 719.2

(51) Int. Cl. A61K 31/496; C07D 491/04

(52) U.S. Cl. 514/252.13; 540/457

(57) ABSTRACT

The present invention relates to novel N-(3-rifamycinyl)-derivatives, namely N-(3-rifamycinyl)-carbamates, methods of their preparation and their use for the production of pharmaceutical preparations. The invention also concerns a composition and a method for treating or preventing mycobacterial infections, especially tuberculosis. Formula (I);

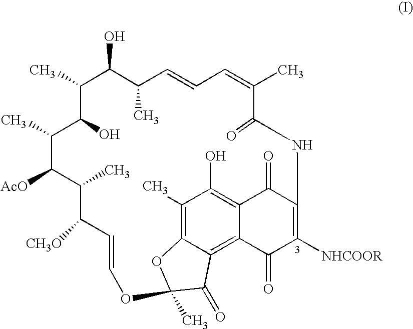

### FIELD OF THE INVENTION

The present invention relates to novel N-(3-rifamycinyl)-derivatives, namely N-(3-rifamycinyl)-carbamates, methods of their preparation and their use for the production of pharmaceutical preparations. The invention also concerns a composition and a method for treating or preventing mycobacterial infections, especially tuberculosis.

### BACKGROUND OF THE INVENTION

Derivatives of rifamycin S or their corresponding hydroquinonic forms rifamycin SV are known to exhibit antibiotic activity against various bacteria by inhibiting the RNA-polymerase, thereby inhibiting synthesis of mRNA.

U.S. Pat. No. 4,005,077, U.S. Pat. No. 4,261,891 U.S. Pat. No. 4,353,826 disclose 3-amino-derivatives derived from rifamycin S and their corresponding hydroquinones derived from rifamycin SV. The compounds may be partially or completely hydrogenated in the rifamycin side chain. According to U.S. Pat. No. 4,353,826 the 3-amino group may be a primary, secondary or tertiary amino group aliphatically linked by hydrocarbon chains which can be interrupted by heteroatoms and/or be substituted by various functional groups. U.S. Pat. No. 4,261,891 shows rifamycin derivatives containing in position 3 an azacycloalkyl group having 2-11 carbon atom in the azacycloalkyl ring and up to 20 carbon atoms at all. In U.S. Pat. No. 4,005,077 the rifamycin S, or rifamycin SV derivatives have a 1-piperazinyl group in position 3 of the rifamycin moiety. The piperazinyl group may be substituted at its N′ position by various groups. The 3-amino-rifamycin-derivatives were shown to exhibit antibiotic activity against gram positive bacteria, particularly against mycobacteria.

### DESCRIPTION OF THE INVENTION

The present invention provides new compounds with anti-mycobacterial activity which are easy to synthesize starting with commercially available substances and which are obtained in good yields. The compounds of the invention have a higher anti-mycobacterial activity than known tuberculosis agents, especially rifampicine. They additionally show anti-microbial activity against ordinary bacteria.

The present invention relates to N-(3-rifamycinyl)-carbamates of the general formula I 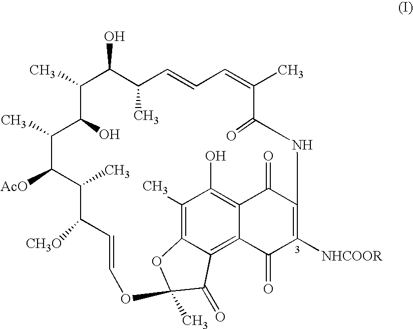
and their corresponding hydroquinones,
wherein R is C₁-C₆-alkyl, mono- or polyhalogenated C₁-C₆-alkyl, C₁-C₆-alkenyl, mono- or polyhalogenated C₁-C₆-alkenyl, triphenylphosphonio-C₁-C₆-alkyl halogenide, menthyl, 9-fluorenylmethyl, piperidyl, or aryl which may be unsubstituted or substituted with one or more of the following groups independently comprising nitro, C₁-C₃-alkoxy, C₁-C₃-alkthio, C₁-C₃-alkoxycarbonyl, di(C₁-C₃-alkylamino), halogen or salts thereof.

In a preferred embodiment the invention relates to compounds according to formula I wherein R is C₁-C₄-alkyl, preferably methyl, ethyl, butyl or isobutyl.

According to another preferred embodiment of the invention R is mono- or polyhalogenated C₁-C₄-alkyl, preferably chloromethyl, 2-chloroethyl, 2-bromoethyl, 2,2,2-trichloroethyl or 2,2,2-trichlor-tert-butyl.

In still another preferred embodiment R is C₁-C₃-alkenyl, preferably vinyl or allyl.

A further preferred embodiment of the invention relates to compounds of formula I wherein R is unsubstituted aryl, preferably benzyl or phenyl.

According to another preferred embodiment R is 4-Nitrobenzyl, 4-Nitrophenyl, 4-methoxycarbonyl phenyl, or 6-nitroveratryl.

Special mention deserve 3-rifamycinyl S-methylcarbamate and 3-rifamycinyl S-ethylcarbamate. These compounds exhibit an in vitro and ex vivo activity against Mycobacterium tuberculosis as well as against various other bacteria (other than mycobacteria) which is at least as high or even higher as the activity of rifampicine.

The novel N-(3-rifamycinyl)-carbamates can be present in the quinonic form (rifamycin S derivatives) and in the hydroquinonic form (rifamycin SV derivatives). Both forms can easily be converted into each other. The compounds may also be present in form of any of their tautomers.

The present invention also encompasses pharmaceutically acceptable salts of the present compounds. Such salts include acid addition salts, metal salts, ammonium and alkylated ammonium salts. Acid addition salts include salts of inorganic acids as well as organic acids.

Representative examples of suitable inorganic acids include hydrochloric, hydrobromic, hydroiodic, phosphoric, sulfuric acids and the like. Representative examples of suitable organic acids include formic, acetic, trichloroacetic, trifluoroacetic, propionic, benzoic, citric, fumaric, glycolic, lactic, maleic, malic, malonic, mandelic, oxalic, picric, pyruvic, salicylic, succinic, methanesulfonic, ethanesulfonic, tartaric acids and the like. Examples of metal salts include lithium, sodium, potassium, magnesium salts and the like. Examples of ammonium and alkylated ammonium salts include ammonium, methyl-, dimethyl-, trimethyl- and tetramethylammonium, ethyl- and diethylammonium, hydroxyethylammonium, butylammonium, salts and the like.

The compounds according to general formula I with R having the aforementioned meanings can easily be prepared by various pathways.

According to one aspect of the invention N-(3-rifamycinyl)-carbamates are prepared by reacting 3-amino rifamycin S of formula II 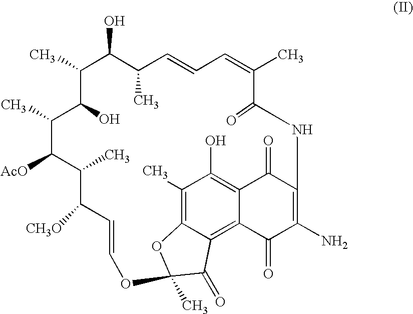
with a chloroformate of formula III 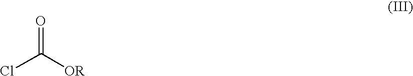
wherein R has the above meanings,
in an organic solvent in the presence of a strong base to give the compound of formula I. In the case the hydroquinone is desired the obtained quinone is subsequently reduced to give the corresponding hydroquinone.

The base is needed for abstracting a proton from the amino group of the 3-amino rifamycin S. According to a preferred embodiment of the invention a tertiary amine, preferably triethylamine or the like, is used as strong base. But also anhydrous sodium carbonate may be used.

Usual organic solvents as for instance dichlormethane, ehtylacetate or tetrahydrofurane can be used for the above reaction. According to the invention it is preferred to use dichloromethane.

The reduction of the quinone product to the corresponding hydroquinone can be done by reducing agents, such as hydrogen sulphite, dithionite or ascorbic acid or its its salts.

This pathway gives suprisingly high yields.

An alternative pathway to synthesize the present compounds starts from 3-formyl rifamycin S according to formula IV 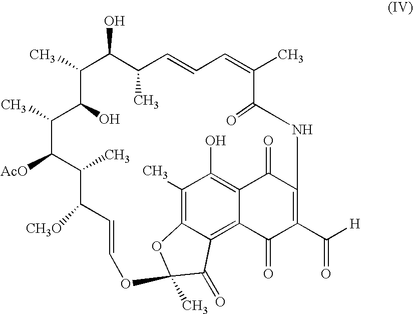
and proceeds via 3-carboxy rifamycin S, via 3-carboxy rifamycin S azide, via the corresponding 3-isocyanate rifamycin S which is formed by reacting the 3-carboxy rifamycin S azide with an alcohol R—OH with R having the above meaning, to finally yield the quinone form according to general formula I. Again the quinonic form can subsequently be converted into the hydroquinonic form if desired.

The products synthesized by both processes are identical according to HPLC retention times and UV-spectra. However, the former pathway is simpler and gives higher yields. Therefore, the pathway starting with 3-amino rifamycin S of formula II is preferred for preparing the present compounds.

The present compounds were shown to have high antibiotic activity against a variety of bacteria, particularly against Mycobacterium tuberculosis and Mycobacterium aurum. Therefore, the invention relates also to the use of N-(3-rifamycinyl)-carbamates of formula I for the production of a pharmaceutical preparation for treating or preventing a mycobacterial infection, particularly for the production of a pharmaceutical preparation for treating or preventing tuberculosis.

In another aspect the invention relates to the use of N-(3-rifamycinyl)-carbamates of formula I for the production of a pharmaceutical preparation for treating or preventing a microbial infection with ordinary bacteria, preferably Bacillus subtilis, Escherichia coli, Bacillus myocide, Klebsiella pneumoniae and/or Pseudomonas aeruginosa. In this connection, the term “ordinary bacteria” relates to others than mycobacterial microorganisms.

In still another aspect the present invention relates to a composition for treating or preventing a mycobacterial and/or an other bacterial infection comprising an anti-mycobacterial and/or anti-bacterial effective amount of at least one compound of formula I or its corresponding hydroquinone with R having the above meaning or a pharmaceutically acceptable salt thereof together with one or more pharmaceutically acceptable carrier(s).

Yet another aspect of the present invention relates to a method for preventing or treating a mycobacterial and/or an other bacterial infection in a mammal comprising administering to a mammal in need of anti-bacterial and/or anti-mycobacterial prevention or treatment an effective anti-mycobacterial amount of at least one compound of formula I or its corresponding hydroquinone, with R having the above meaning or a pharmaceutically acceptable salt thereof and a pharmaceutically acceptable carrier therefore.

The pharmaceutical compositions according to the invention may be formulated with pharmaceutically acceptable carriers or diluents as well as other known adjuvants and excipients in accordance with conventional techniques.

The pharmaceutical compositions may be specifically formulated for administration by any suitable way such as oral, rectal, nasal, pulmonary, topical (including buccal and sublingual), transdermal, intracisternal, intraperitoneal, vaginal, and par enteral (including subcutaneous, intramuscular, intrathecal, intravenous, and intradermal) route. It will be appreciated that the preferred route will depend on the general condition and age of the subject to be treated, the nature of the disorder to be treated and the active agent chosen.

Pharmaceutical compositions for oral administration include solid dosage forms such as capsules, tablets, dragees, pills, lozenges, powders, and granules. Where appropriate, they can be prepared with coatings such as enteric coatings or they can be formulated as to provide controlled release of the active ingredient such as prolonged release according to well-known methods.

Liquid dosage forms for oral administration include solutions, mulsions, suspensions, syrups and elixirs.

Pharmaceutical compositions for parental administration include sterile aqueous and non-aqueous injectable solutions, dispersions, suspensions or mulsions as well as sterile powders to be reconstituted in sterile injectable solutions or dispersions prior to use.

Other suitable administration forms include suppositories, sprays, ointments, cremes, gels, inhalants, dermal patches, implants and the like.

A typical oral dosage is in the range of from about 0.001 to about 100 mg/kg body weight per day, preferably from about 0.01 to about 50 mg/kg body weight, and more preferred from about 0.05 to about 10 mg/kg body weight per day administered in one or more dosages, such as 1 to 3 dosages. It is understood that the exact dosage will depend on the frequency and mode of administration, the sex, age, weight and general condition of the subject treated, the nature and severity of the condition treated and concomitant diseases to be treated and other factors evident to those skilled in the art.

Accordingly, the pharmaceutical compositions according to the invention for oral administration one or more times per day comprise at least one of the compounds according to formula I from about 0.05 mg to about 1000 mg, preferably from about 0.1 mg to about 500 mg, especially preferred from about 1 mg to about 200 mg of the compound. For parenteral routes, such as intravenous, intrathecal, intramuscular and the like, typical doses are in the order of about half the dose employed for oral administration.

Suitable pharmaceutical carriers include inert solid diluents or filles, sterile aqueous solution and various organic solvents. Examples of solid carriers are lactose, terra alba, sucrose, cyclodextrin, talc, gelatine, agar, pectin, acacia, magnesium stearate, stearic acid or lower alkyl ethers of cellulose. Examples of liquid carriers are syrup, peanut oil, phospholipids, fatty acids, fatty acid amines, polyoxyethylene or water. Similarly, the carrier or diluent may include any sustained release material known in the art, such as glyceryl monostearate or glyceryl distearate, alone or mixed with wax.

### EXAMPLES

The present invention is further illustrated by the following representative examples which are not intended to limit the scope of the invention in any way.

### Example 1

### N-(Ethyloxycarbonyl)-3-aminorifamycin S

A solution of 10 g 3-aminorifamycin S and 3 ml triethylamine in 100 ml dichloromethane was cooled to −5° C. and 1.5 ml ethylchloroformate were added. The solution was stored at room temperature for 24 hours and 2 ml triethylamine and 1 ml ethylchloroformate were added.

After storage at room temperature for additional 2 hours the solution was evaporated under reduced pressure to yield an oily residue. 150 ml tetrachlormethane and 100 ml 10% ammonium chloride/water solution were added and the mixture was stirred for one hour. The emulsion was filtered off, the cake was washed by 60 ml tetrachloromethane and 150 water and to the filtrate 150 ml hexane were added. After stirring for 15 minutes the product was filtered off, washed with water and dried. 6 g of pink crystals were yielded.

Structural analysis of the product was done by HPLC, TLC, UV spectroscopy, IR spectroscopy and NMR spectroscopy (¹H¹³C and DEPT). ¹H-NMR spectroscopy on a Bruker drx250 in a solution of CDCl₃ and (CD₃)₂SO gave the following spectrum:

8.26 ppm, 1H, NH—CO; 6.4-6.05 ppm, m, 7H; 6.45 ppm, dd, 1H, J=15.8; 10.4, H-18; 6.25 ppm, d, 1H, 7=9.6, H-17; 6.14 ppm, dd, 1H, J=17.3; 6.8, H-19; 6.07 ppm, d, 1H, J=13.2, H-29; 6.05 ppm, br.s, 1H; 5.1 ppm, dd, 1H, J=12.4; 5.5, H-28; 4.97 ppm, d, 1H, J=10.4, H-25; 4.40 q, 2H, J=7.1, CH₂CH₃; 3.91 ppm, d, 1H, J=5.1, OH-21; 3.75 ppm, d, 1H, J=9.5, H-21; 3.09 ppm, m, 1H, H-23; 3.07 ppm, s, 3H, H-37; 2.35 ppm, s, 3H, H-36; 2.3 ppm, 1H, H-22; 2.0-1.9 ppm, 7H, H-14, H-30, NH or CH; 1.78 ppm, s, 3H, H-13; 1.7-1.5, m, 1H, H-24; 1.43 ppm, t, 3H, J=7.1, CH₂CH₃; 1.03 ppm, d, 3H, J=7.0, H-31; 0.85 ppm, d, 3H, J=6.9, H-32; 0.66 ppm, d, 3H, J=6.8, H-33; 0.1 ppm, d, 3H, J=6.9, H-34.

### Example 2

### N-(Ethyloxycarbonyl)-3-aminorifamycin S

To the solution of 12 g 3-formylrifamycin S in 100 ml tetrahydrofurane 4 ml triethylamine. and 8 g silver(l) oxide were added. The mixture was stirred at room temperature for 18 hours and 250 ml dichloromethane and 500 ml 4% ammonium chloride/water solution were added. After stirring for 15 minutes the mixture was filtrated, the dichloromethane layer was separated, dried over anhydrous sodium sulfate and evaporated to dryness. The residue was dissolved in 100 ml tetrahydrofurane, the solution was cooled to 0 ° C. and 5 ml diphenylphosphoryl azide were added. The solution was stored at 0 ° C. for 8 hours and 5 ml absolute ethanole were added. The solution was heated at 60 ° C. for 5 hours and evaporated to an oily residue. After column chromatography on silicagel 60 (70-230 mesh) with mobile phase chloroform: acetone 5:1 the violet fraction was evaporated and the product was crystallized in tetrachloromethane—hexane, filtered and dried. 1.8 g of pink crystals were yielded.

The product is identical to that from example 1 as proved by retention times according to various HPLC and TLC methods and by UV-spectra (HPLC).

### Example 3

### N-(Methyloxycarbonyl)-3-aminorifamycin S

A solution of 10 g 3-aminorifamycin S and 4 ml triethylamine in 100 ml dichloromethane was cooled to −5° C. and 1.5 ml methylchloroformate were added. The solution was stored at room temperature for 40 hours and 2 ml triethylamine and 1 ml methylchloroformate were added. After additional 5 hours at room temperature the solvent was evaporated in vacuum. 450 ml tetrachloromethane and 100 ml 10% ammonium chloride/water solution were added and, after stirring for an hour the mixture was filtered of and the cake washed with tetrachloromethane and water. 450 ml hexane were added and after stirring for 15 minutes the product was filtered, washed with water and dried. 7 g of violet crystals were yielded. Structural analysis of the product was done by HPLC, TLC, UV spectroscopy, IR spectroscopy and NMR. spectroscopy (¹H¹³C and DEPT).

### Example 4

### N-(4-Nitrobenzyloxycarbonyl)-3-aminorifamicin S

To a solution of 10 g 3-aminorifamycin S and 7 ml triethylamine in 100 ml dichloromethane, cooled to −20° C., 6 g 4-nitrobenzylchloroformate were added. The solution was stored at 0° C. for 3 hours and the solvent was evaporated in vacuo. To the residue 250 ml tetrachloromethane and 200 ml 5% ammonium chloride/water solution were added and the mixture was stirred for 1 hour. After filtration and washing of the cake with tetrachloromethane to the filtrate 300 ml hexane were added. The mixture was stored at 0° C. overnight and the product was filtered, washed with water and dried. 6 g of dark pink crystals were yielded. Structural analysis of the product was done by HPLC, TLC, UV spectroscopy, IR spectroscopy and NMR spectroscopy (¹H¹³C and DEPT).

### Example 5

### N-(2-Bromoethyloxycarbonyl)-3-aminorifamicin S

To a solution of 10 g 3-amino rifamycin S and 6,8 ml triethylamine in 100 ml dichloromethane cooled to −20° C. 2,6 ml 2-bromoethylchloroformate were added. The solution was stored at −5° C. for 1.5 hors and the solvent was evaporated in vacuum. To the residue 350 ml tetrachloromethane and 100 ml 15% ammonium chloride/water solution were added and the mixture stirred for an hour. The suspension was filtered off, to the filtrate 500 ml hexane were added and, after storage of the mixture at −50° C. overnight, the product was filtered, washed with water and dried. 6.5 g of dark pink crystals were yielded. Structural analysis of the product was done by HPLC, TLC, UV spectroscopy, IR spectroscopy and NMR spectroscopy (¹H¹³C and DEPT).

### Example 6

### In vitro testing for Anti-Microbacterial Activity (Bacteria other than Mycobacteria)

The compounds according to the present invention with R=methyl, ethyl, 2-bromoethyl and 4-nitrobenzyl were tested in vitro against some representative strains of ordinary (non-mycobacterial) bacteria in comparison with rifampicin (3-[4-methyl-1-piperainylimino)-methyl]-rifamycin), using double-dosage method of diffusion in agar. This test is based on the logarithmic dependence between the size of the inhibition area associated with the bacterial growth in a layer of agar (response) and the quantity of the applied antibiotic.

The reference substance (rifampicin) and the tested compounds were dissolved in methanol to give concentrations of 1 mg/ml. From these solutions buffered solutions were prepared in phosphate buffer, pH 7.4 at concentrations of 5 and 10 μg/ml.

An initial suspension of the test microorganism (Bacillus subtilis ATCC 6633, Escherichia coli, Bacillus mycoide, Klebsiella pneumoniae, Pseudomonas aeruginosa) was prepared having a UV-light transmission of about 25%. A suitable diffusion medium (for example for Bacillus subtilis: 1 g pepton, 3 g yeast extract, 15-18 g agar, in 1 l water, pH 7.8-8.0 after sterilization) was inoculated with 0.5 ml of the initial suspension per 100 ml medium at a temperature of 60-65° C. 20 ml of the mixture each was filled into Petri's dishes (diameter 100 mm). After hardening of the medium four pits (diameter 5 mm) were placed in each dish and onto every pit 90 μl of the solutions of the reference and the tested compounds were added. The dishes were incubated at 37° C. for at least 15 hours. The sizes (diameters) of the areas of inhibition were then measured with an accuracy of 0.1 mm.

The activity A was calculated according to
lg A=(lv/w) with
I=lg (high concentration/low concentration),
v=(ΣX₁+ΣX₂)−(ΣP₁+ΣP₂), and
w=(ΣX₂+ΣP₂)−(ΣX₁+ΣP₁),
   with
X₁: area size in mm at low concentration of the sample,
X₂: area size in mm at high concentration of the sample,
P₁: area size in mm at low concentration of the reference,
P₂: area size in mm at low concentration of the reference,

The relative activities are shown in Table 1 (the activity of rifampicin is taken as 1000). Against Bacillus subtilis all of the four tested compounds showed antibiotic activities higher than the reference rifampicine. Especially the substances with R=methyl, 2-bromoethyl and 4-nitrobenzyl exhibited activities being about six times higher than rifampicine.

The compounds with R=methyl and ethyl were tested against E. coli, B. subtilis, K. pneumoniae and P. aeruginosa. Against these strains N-(methyloxycarbonyl)-3-aminorifamycin S showed an activity twice as high as rifampicine, whereas N-(ethyloxycarbonyl)-3-aminorifamycin S has similar activities as the reference.

TABLE 1

| | Bacillus | | | | |
|---|---|---|---|---|---|
| | subtilis | | | | |
| | ATCC | Escherichia | Bacillus | Klebsiella | Pseudomonas |
| R | 6633 | coli | mycoide | pneumoniae | aeruginosa |
| methyl | 6000 | 2100 | 2000 | 1900 | 1750 |
| ethyl | 2050 | 690 | 1150 | 1030 | 920 |
| 2- | 6600 | — | — | — | — |
| bromo- | | | | | |
| ethyl | | | | | |
| 4-nitro- | 5400 | — | — | — | — |
| benzyl | | | | | |

### Example 7

### Ex vivo Testing for Anti-Mycobacterial Activity—Growth in Mouse Macrophages

Because of the possibility that compounds become concentrated in macrophages and because mycobacteria are intracellular pathogens, the intracellular activity of compounds was determined. This was achieved by addition of the compounds to the mouse macrophage cell line J774, that had been infected with M. tuberculosis H37Rv. The activity of the compounds was then measured by determining the number of colony forming units present in each monolayer and culture medium.

In detail, mouse macrophage cell line J774 was obtained from the European Collection of Animal Cell Culture and stored in liquid nitrogen. J774 cells were grown in RPMI 1640 medium supplemented with 1 mM L-glutamine and 10% (v/v) heat-inactivated foctal bovine serum [HIFBS] at 37° C. and 5% (v/v) CO₂. When a confluent monolayer had formed on the surface of the tissue culture flask, the cells were subcultured. The medium was removed; the cells were washed twice in 10 ml of HBSS-Hepes and 2 ml of trypsin-EDTA solution was added to the monolayer. After incubation of the monolayer at 37° C. and 5% (v/v) CO₂ the cells were removed from the surface by sharp tapping on the flask. 20 ml of fresh RPMI 1640 medium plus HIFBS was added to the flask and transferred to a centrifuge tube and centrifuged at 1.000 rpm for 5 minutes in a Centaur 2 MSE centrifuge to remove traces of trypsin-EDTA. The medium was removed and 1 ml fresh RPMI 1640 medium plus HIFBS was added and the cells were pipetted gently to separate clumps. 300 μl of the cell suspension was added to 10 ml RPMI 1640 medium plus HIFBS in a new tissue culture flask and the cells were incubated at 37° C. and 5% (v/v) CO₂. To count numbers of viable macrophages, 20 μl of the cell suspension was added to 40 μl of 0.2% (v/v) trypan blue in Hanks balanced salt solution (calcium and magnesium free without phenol red). 20 μl of this solution was then transferred to a chamber of a haemocytometer and the cells were counted. Viable cells remained unstained and white in color and dead cells stained blue.

Stock cultures of Mycobacterium tuberculosis H37Rv were maintained on Middlebrook 7H11 agar+OADC plates or on Lowenstein-Jensen [LJ] agar slopes for up to one month at 4° C. [7H11 plates] or for up to 6 months at −20 ° C. [LJ slopes]. The challenge dose was a culture in Middlebrook 7119 broth supplemented with ADC that had been incubated at 37° C. for 7 days. The bacteria were harvested by centrifugation at 1.000 g for 10 minutes and then washed twice in HBSS-Hepes pH 7.4. The cell pellets were resuspended in 1 ml of HBSS-Hepes and sonicated on ice for three 5 s bursts at 40 W to disrupt clumps of bacteria. The mycobacteria were counted microscopically using haemocytometer and then were diluted in RPMI 1640 medium plus 1% (v/v) HIFBS.

To prepare monolayers for infection, J774 cells were removed from the tissue culture flask and counted using a heamocytometer. The trypan blue exclusion assay was used to determine viability as described above and 3×10⁷ cells in a volume of 350 μl RPMI 1640 medium plus 10% (v/v) HIFBS were pipetted into each well of a 24 well tissue culture plate. The cells were incubated for 24 hours at 37° C. and 5% (v/v) CO₂ to enable adherence of the cells to the surface of the wells. After 24 hours, non-adherent cells were removed by washing once with HBSS-Hepes. The resulting macrophage monolayer was cultured in RPMI 1640 medium plus 1% (v/v) HIFBS to reduce cell proliferation.

Mycobacterium tuberculosis was diluted in RPMI 1640 medium plus 1% (v/v) HIFBS to obtain a 1:1 ratio of mycobacteria to macrophage. 350 μl of medium containing 3×10⁷ bacteria were gently added into the wells of the 24 well tissue culture plate containing the adherent J774s and, incubated for 4 hours at 37° C. and 5% (v/v) CO₂ to allow phagocytosis. The supernatant was aspirated and the monolayer was washed four times in HBSS-Hepes to remove unphagocytosed mycobacteria. Fresh RPMI 1640 medium plus 1% (v/v) HIFBS with or without test compound was added to the macrophages. Macrophages were incubated for 24 hours at 37° C. and 5% (V/V) CO₂.

The supernatants from each well of the 24 well tissue culture plate then were removed and set-aside. The macrophages were removed from the wells of the plate by addition of 350 μl of 1% (w/v) saponin in HBSS-Hepes. 35 μl of 10% (w/v) saponin in HBSS-Hepes was added to the supernatants. The 24 well tissue culture plate and supernatants were incubated at 37° C. for 20 minutes or until the macrophages had completely lysed and then were mixed by pipetting up and down. Cell lysis was checked microscopically using a Nikon inverted microscope. Cell lysates and supernatant were briefly sonicated on ice for three 5 s bursts at 40 W to ensure complete cell lysis and disruption of any bacterial clumps. Viable counts were performed to estimate the number of extracellular and intracellular bacteria as described in example 8.

As can be seen from Table 2, both tested compounds were remarkably effective in this assay. In particular, they showed activities 8 times higher than rifampicine.

TABLE 2

| | Macrophage Data (CFU/ml) | | | | |
|---|---|---|---|---|---|
| | Monolayer | Monolayer | Monolayer | Monolayer | Monolayer |
| Compound | 1 | 2 | 3 | 4 | mean |
| rifampicine | 0.00 | 200.00 | 600.00 | 800.00 | 400.00 |
| R = ethyl | 0.00 | 200.00 | 0.00 | 0.00 | 50.00 |
| R = methyl | 0.00 | 0.00 | 0.00 | 200.00 | 50.00 |
| DMSO | 1.0 * 10⁶ | 8.6 * 10⁶ | 10.8 * 10⁶ | 7.4 * 10⁶ | 9.3 * 10⁶ |

1. N-(3-rifamycinyl)-carbamates of the formula I 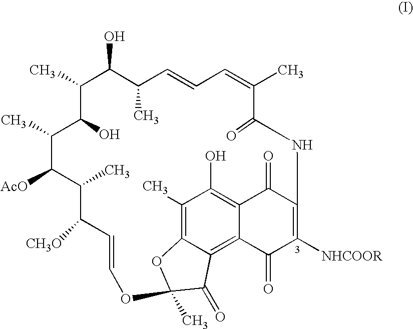
and their corresponding hydroquinones,
wherein R is C₁-C₆-alkyl, mono- or polyhalogenated C₁-C₆-alkyl, C₁-C₆-alkenyl, mono- or polyhalogenated C₁-C₆-alkenyl, triphenylphosphonio-C₁-C₆-alkyl halogenide, menthyl, 9-fluorenylmethyl, piperidyl, or aryl which may be unsubstituted or substituted with one or more of the following groups independently comprising nitro, C₁-C₃-alkoxy, C₁-C₃-alkylthio, C₁-C₃-alkoxycarbonyl, di(C₁-C₃-alkylamino), halogen or salts thereof.

2. Carbamates of claim 1, wherein
R is C₁-C₄-alkyl, preferably methyl, ethyl, butyl or isobutyl.

3. Carbamates of claim 1, wherein
R is mono- or polyhalogenated C₁-C₄-alkyl, preferably chloromethyl, 2-chloroethyl, 2-bromoethyl, 2,2,2-trichloroethyl or 2,2,2-trichlor-tert-butyl.

4. Carbamates of claim 1, wherein
R is C₁-C₃-alkenyl, preferably vinyl or allyl.

5. Carbamates of claim 1, wherein R is unsubstituted aryl, preferably benzyl or phenyl.

6. Carbamates of claim 1, wherein
R is 4-Nitrobenzyl, 4-Nitrophenyl, 4-methoxycarbonyl phenyl, or 6-nitroveratryl.

7. A method of preparing a N-(3-rifamycinyl)-carbamate according to formula I 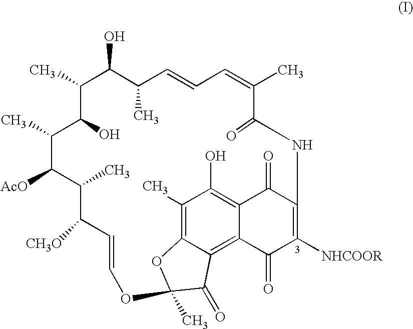
and their corresponding hydroquinones,
wherein R is C₁-C₆-alkyl, mono- or polyhalogenated C₁-C₆-alkyl, C₁-C₆-alkenyl, mono- or polyhalogenated C₁-C₆-alkenyl, triphenylphosphonio-C₁-C₆-alkyl halogenide, menthyl, 9-fluorenylmethyl, piperidyl, or aryl which may be unsubstituted or substituted with one or more of the following groups independently comprising nitro, C₁-C₃-alkoxy, C₁-C₃-alkylthio, C₁-C₃-alkoxycarbonyl, di(C₁-C₃-alkylamino), halogen
characterized in that 3-amino rifamycin S of formula II 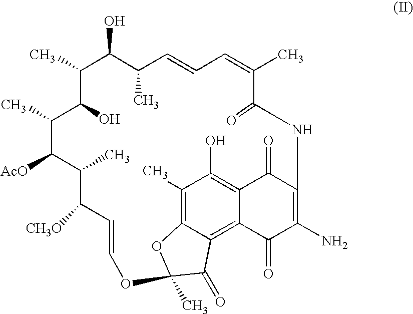
is reacted with a chloroformate of formula III 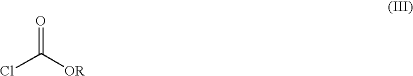
wherein R has the above meanings,
in an organic solvent in the presence of a strong base, and optionally the obtained quinone compound of formula I is reduced to give the corresponding hydroquinone.

8. The method according to claim 7,
characterized in that
as a strong base a tertiary amine, preferably triethylamine is used.

9. The method according to claim 7,
characterized in that
as organic solvent dichloromethane, ethylacetate or tetrahydrofurane is used.

10. Use of N-(3-rifamycinyl)-carbamates of formula I of claim 1 for treating or preventing a mycobacterial infection.

11. Use of N-(3-rifamycinyl)-carbamates of formula I of claim 1 for the production of a pharmaceutical preparation for treating or preventing a mycobacterial infection.

12. Use of compounds according to claim 10 for treating or preventing tuberculosis.

13. Use of compounds according to claim 11 for the production of a pharmaceutical preparation for treating and preventing tuberculosis.

14. Use of N-(3-rifamycinyl) carbamates of formula I of claim 1 for the production of a pharmaceutical preparation for treating or preventing a microbial infection with ordinary (non-mycobacterial) bacteria, preferably Bacillus subtilis, Escherichia coli, Bacillus myocide, Klebsiella pneumoniae and/or Pseudomonas aeruginosa.

15. Use of N-(3-rifamycinyl) carbamates of formula I of claim 1 for treating or preventing a microbial infection with ordinary (non-mycobacterial) bacteria, preferably Bacillus subtilis, Escherichia coli, Bacillus myocide, Klebsiella pneumoniae and/or Pseudomonas aeruginosa.

16. A composition for treating or preventing a mycobacterial infection and/or a microbial infection with ordinary (non-mycobacterial) bacteria comprising an anti-mycobacterial and/or anti-bacterial effective amount of a compound of formula I 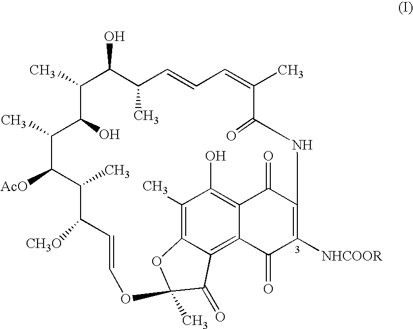
or its corresponding hydroquinone,
wherein R is C₁-C₆-alkyl, mono- or polyhalogenated C₁-C₆-alkyl, C₁-C₆-alkenyl, mono- or polyhalogenated C₁-C₆-alkenyl, triphenylphosphonio-C₁-C₆-alkyl halogenide, menthyl, 9-fluorenylmethyl, piperidyl, or aryl which may be unsubstituted or substituted with one or more of the following groups independently comprising nitro, C₁-C₃-alkoxy, C₁-C₃-alkylthio, C₁-C₃-alkoxycarbonyl, di(C₁-C₃-alkylamino), halogen
or a pharmaceutically acceptable salt thereof
and a pharmaceutically acceptable carrier therefore.

17. A composition according to claim 16 comprising from about 0.05 mg to about 1000 mg, preferably from about 0.1 mg to about 500 mg, especially preferred from about 1 mg to about 200 mg of the compound according to formula I.

18. A method for preventing or treating a mycobacterial infection and/or a microbial infection with ordinary (non-mycobacterial) bacteria in a mammal comprising administering to a mammal in need of anti-mycobacterial and/or anti-bacterial prevention or treatment an effective anti-mycobacterial and/or antibacterial amount of at least one compound of formula I 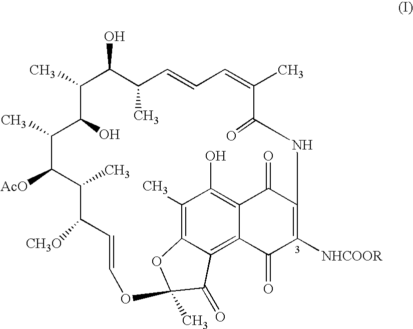
or its corresponding hydroquinone,
wherein R is C₁-C₆-alkyl, mono- or polyhalogenated C₁-C₆-alkyl, C₁-C₆-alkenyl, mono- or polyhalogenated C₁-C₆-alkenyl, triphenylphosphonio-C₁-C₆-alkyl halogenide, menthyl, 9-fluorenylmethyl, piperidyl, or aryl which may be unsubstituted or substituted with one or more of the following groups independently comprising nitro, C₁-C₃-alkoxy, C₁-C₃-alkylthio, C₁-C₃-alkoxycarbonyl, di(C₁-C₃-alkylamino), halogen
or a pharmaceutically acceptable salt thereof
and a pharmaceutically acceptable carrier therefore.
